(19) United States
(12) Patent Application Publication
(10) Pub. No.: US 2022/0192177 A1
(43) Pub. Date: Jun. 23, 2022

(54) SYSTEMS AND METHODS FOR PERFUSING A HUMAN PLACENTA-BASED MRI PHANTOM

(71) Applicants: Children's Medical Center Corporation, Boston, MA (US); Massachusetts Institute of Technology, Cambridge, MA (US); The General Hospital Corporation, Boston, MA (US)

(72) Inventors: Patricia Ellen Grant, Newton, MA (US); Drucilla Roberts, Millis, MA (US); Esra Abaci Turk, Boston, MA (US); Jeffrey N. Stout, Jamaica Plain, MA (US); Lawrence L. Wald, Cambridge, MA (US); Elfar Adalsteinsson, Belmont, MA (US); William Barth, Boston, MA (US)

(73) Assignees: Children's Medical Center Corporation, Boston, MA (US); Massachusetts Institute of Technology, Cambridge, MA (US); The General Hospital Corporation, Boston, MA (US)

(21) Appl. No.: 17/553,999

(22) Filed: Dec. 17, 2021

Related U.S. Application Data
(60) Provisional application No. 63/127,953, filed on Dec. 18, 2020.

(51) Int. Cl. A01N 1/02; A61B 5/055; G01R 33/30; G01R 33/58

(52) U.S. Cl. CPC A01N 1/021; G01R 33/58; G01R 33/30; A61B 5/055

(57) ABSTRACT

Provided herein are systems and methods for development and use of a perfusion apparatus comprising a biological phantom created from an ex vivo placenta. In some embodiments, a system is provided for perfusing an ex vivo placenta to be imaged using a magnetic resonance imaging (MRI) device, the system comprising a chamber configured to house the ex vivo placenta therein, the chamber including a first partition separating the chamber into a first portion and a second portion, wherein the ex vivo placenta is housed at least partially in the first portion, and at least one first inlet disposed in the second portion for receiving at least one first tube, the at least one first tube being configured to couple at least one first pump to a fetal compartment of the ex vivo placenta when present in the chamber.

### CROSS-REFERENCE TO RELATED APPLICATIONS

This application claims the benefit under 35 U.S.C. § 119(e) of U.S. Provisional Patent Application Ser. No. 63/127,953, filed Dec. 18, 2020, under Attorney Docket No. C1233.70192US00, and entitled “SYSTEMS AND METHODS FOR PERFUSING A HUMAN PLACENTA-BASED MRI PHANTOM,” which is hereby incorporated by reference herein in its entirety.

### FEDERALLY SPONSORED RESEARCH

This invention was made with government support under Grant No. R01EB017337, Grant No. R01HD100009, and Grant No. U01HD087211 awarded by the National Institutes of Health. The government has certain rights in the invention.

### BACKGROUND

Magnetic resonance imaging (MRI) is a non-invasive and versatile technique for studying the physiology and pathology of biological systems. Generally, MRI operates by detecting magnetic resonance (MR) signals emitted by the nuclei of atoms in a subject in response to changes in magnetic fields and applied electromagnetic radiation (e.g., radio waves). The detected MR signals may then be used to generate MR images of the subject.

In some instances, a phantom may be used to replicate an aspect of an object that would be scanned using MRI, but in a form that is reproducible and easy to manipulate. Phantoms are typically developed from synthetic material, such as a mixture of plastic and agar, and are designed to replicate the properties of a patient anatomy such that imaging results with a phantom are comparable to imaging the patient anatomy itself.

Perfusion refers to the passage of blood or other fluid through vessels or other channels in an organ or tissue. Perfusion phantoms have been developed which mimic fluid flow through organs, both to standardize measurements between MRI scanners and to develop new imaging technology to more accurately quantify perfusion.

### SUMMARY

Some embodiments provide for a system for perfusing an ex vivo placenta to be imaged using a magnetic resonance imaging (MRI) device, the system comprising: a chamber configured to house the ex vivo placenta therein, wherein the chamber includes a first partition separating the chamber into a first portion and a second portion, wherein the ex vivo placenta is housed at least partially in the first portion; and at least one first inlet disposed in the second portion for receiving at least one first tube, the at least one first tube being configured to couple at least one first pump to a fetal compartment of the ex vivo placenta when present in the chamber.

Some embodiments provide for a method for perfusing an ex vivo placenta to be imaged using a magnetic resonance imaging (MRI) device, the ex vivo placenta being disposed in a chamber, the method comprising: pumping, using at least one first pump, a solution through at least one first tube to a fetal compartment of the ex vivo placenta; and imaging, using the MRI device, the ex vivo placenta as the solution is pumped through the at least one first tube.

Some embodiments provide for a magnetic resonance imaging (MRI) compatible perfusion apparatus comprising: a chamber configured to house an ex vivo placenta therein, the chamber comprising: at least one first inlet arranged to receive at least one first tube configured to couple to a fetal compartment of the ex vivo placenta when present in the chamber; and at least one second inlet configured to receive at least one second tube configured to couple to a maternal compartment of the ex vivo placenta when present in the chamber; and at least one radio frequency (RF) coil arranged proximate to the chamber and configured transmit RF signals and/or detect MR signals generated, at least in part, by the ex vivo placenta when present in the chamber during MR imaging.

Some embodiments provide for a method for generating at least one magnetic resonance (MR) image of an ex vivo placenta disposed in a chamber, the method comprising: perfusing the ex vivo placenta while the chamber is located proximate to at least one radio frequency (RF) coil of a magnetic resonance imaging (MRI) system; transmitting, using the at least one RF coil, at least one RF signal; detecting, using the at least one RF coil, at least one MR signal generated, at least in part, by the ex vivo placenta present in the chamber in response to stimulation of the ex vivo placenta by the at least one RF signal; and generating, an MR image based on the at least one MR signal detected by the at least one RF coil.

### BRIEF DESCRIPTION OF DRAWINGS

Various aspects and embodiments of the disclosed technology will be described with reference to the following figures. It should be appreciated that the figures are not necessarily drawn to scale. For purposes of clarity, not every component may be labeled in every drawing.

FIG. 1 is a schematic diagram of an example system for imaging an ex vivo placenta using a magnetic resonance imaging device, in accordance with some embodiments described herein.

FIG. 2A illustrates an example apparatus for perfusing an ex vivo placenta, in accordance with some embodiments of the technology described herein.

FIG. 2B illustrates a cross-sectional view of the example apparatus of FIG. 2A, in accordance with some embodiments of the technology described herein.

FIG. 2C illustrates an example radio-frequency coil of the example apparatus of FIG. 2A, in accordance with some embodiments of the technology described herein.

FIG. 3 illustrates an example system for perfusing an ex vivo placenta, in accordance with some embodiments of the technology described herein.

FIG. 4 illustrates additional aspects of the example system of FIG. 3, in accordance with some embodiments of the technology described herein.

FIG. 5A illustrates an example process for perfusing an ex vivo placenta, in accordance with some embodiments of the technology described herein.

FIG. 5B illustrates an example process for generating at least one magnetic resonance image of an ex vivo placenta, in accordance with some embodiments of the technology described herein.

FIG. 5C illustrates an example timing diagram for alternating between pump on and pump off states, in accordance with some embodiments of the technology described herein.

FIG. 6A illustrates an example of an ex vivo placenta, in accordance with some embodiments of the technology described herein.

FIG. 6B illustrates an example of a perfused ex vivo placenta, in accordance with some embodiments of the technology described herein.

FIG. 6C illustrates an example magnetic resonance angiography maximum intensity projection of the ex vivo placenta of FIG. 6A, in accordance with some embodiments of the technology described herein.

FIG. 6D illustrates an example histological examination of portions of the ex vivo placenta of FIG. 6A, in accordance with some embodiments of the technology described herein.

FIG. 6E illustrates an example T2 map of the ex vivo placenta of FIG. 6A.

FIG. 6F illustrates an example T1 map of the ex vivo placenta of FIG. 6A.

FIGS. 7A-7B illustrate example contrast enhanced magnetic resonance angiography projections of an ex vivo placenta captured using imaging of a biological placental perfusion device configured in accordance with some embodiments of the technology described herein.

FIG. 7C illustrates an example ex vivo placenta after a washout has been performed that may be used with a biological placental perfusion device configured in accordance with some embodiments of the technology described herein.

FIG. 7D illustrates the example ex vivo placenta of FIG. 7C after magnetic resonance imaging has been performed.

FIG. 7E illustrates an example histological examination of portions of the ex vivo placenta of FIG. 7C.

FIGS. 8A-8B illustrate example images of a perfused ex vivo placenta captured using imaging of a biological placental perfusion device configured in accordance with some embodiments of the technology described herein.

FIGS. 9A-9B illustrate images of an ex vivo placenta having infarcted regions captured using imaging of a biological placental perfusion device configured in accordance with some embodiments of the technology described herein.

FIG. 9C illustrates an example T1 map of the ex vivo placenta of FIGS. 9A-9B, in accordance with some embodiments of the technology described herein.

FIG. 9D illustrates an example T2 maps of the ex vivo placenta of FIGS. 9A-9B.

FIG. 10 illustrates example magnetic resonance angiography data during perfusion of an intervillous space and umbilical artery of an ex-vivo placenta captured using imaging of a biological placental perfusion device configured in accordance with some embodiments of the technology described herein.

FIG. 11A illustrates sine coronal maximum intensity projections of four perfused ex vivo placentas determined based on imaging data captured using imaging of a biological placental perfusion device configured in accordance with some embodiments of the technology described herein.

FIG. 11B illustrates example correlation plots between magnetic resonance fingerprinting for the projections of FIG. 11A and reference techniques for T1 and T2.

FIG. 11C illustrates example T1 and T2 graphs of the ex vivo placentas of FIG. 11A.

FIG. 12A illustrates example magnetic resonance images obtained during perfusion of maternal compartments of a pair of ex vivo placentas with the biological placental perfusion device configured in accordance with some embodiments of the technology described herein.

FIG. 12B illustrates the example magnetic resonance images of FIG. 12A, having

FIG. 12C illustrates example T1 and T2 graphs of the ex vivo placentas of FIG. 12A.

FIG. 13 illustrates a block diagram of an example computer system, in accordance with some embodiments of the technology described herein.

### DETAILED DESCRIPTION

Aspects of the present application relate to systems and methods for development and use of a perfusion apparatus comprising a biological phantom created from an ex vivo placenta. In some embodiments, the perfusion apparatus may be used to develop MR scanning techniques, including, for example, to estimate flow sensitivity of magnetic resonance fingerprinting (MRF).

The placenta is the site of exchange of oxygen between the mother and the fetus, in particular, between maternal and fetal compartments of the placenta. MRI (e.g., relaxometry, including MRF) may be used to monitor placental function, for example, in cases of preeclampsia or fetal growth restriction, as well as to provide information on tissue microstructure of the placenta. The vascular pathways of the placenta, however, are complex, which makes the placenta difficult and time-consuming to synthetically replicate.

In addition to its complicated structure, the placenta experiences a large percentage blood flow by volume (e.g., 50%) in comparison to the other anatomy such as the brain with about 4% blood flow by volume. Although it is known that the placenta experiences a large volume of blood flow, the precise percentage of blood by volume is unknown. Further, the percentage of blood flow by volume in each compartment of the placenta is also unknown. Each of these issues further complicates synthetically replicating the placenta.

Due to these issues, developing an artificial model to accurately mimic the effects of blood flow in a human placenta has not been achieved, and as such the effect of very large flowing blood volume on developmental MRI sequences that quantify relaxation parameters was previously unknown. The inventors have recognized, however, that the placenta is unique among other patient anatomy. Although it is a vital organ, the placenta becomes disposable after a period of time. Given that no other organ is unique in this way, use of a human biological specimen in a controlled environment as a phantom to test MRI techniques has not previously been contemplated, nor has the challenge of making an ex vivo specimen MRI compatible been addressed.

The inventors have developed techniques for using an ex vivo placenta as a biological phantom. The systems and methods described herein provide for a perfusion apparatus that is MRI compatible and capable of perfusing an ex vivo placenta. The perfusion apparatus described herein may independently modulate perfusion of both the maternal and fetal compartments of the ex vivo placenta, making it a powerful tool for testing imaging sequences against the different perfusion characteristics of each compartment. The inventors have recognized that the placental perfusion phantom described herein more precisely mimics biological perfusion than any previous phantom and as such may be used to develop and validate MRI perfusion quantification and to examine the confounding effects of perfusion on quantitative MRI techniques.

Aspects of the present disclosure relate to systems and methods for perfusing a human placenta-based MRI phantom. According to some aspects of the technology described herein, there is provided a system for perfusing an ex vivo placenta to be imaged using an MRI device, the system comprising: (1) a chamber configured to house the ex vivo placenta therein, wherein the chamber includes a first partition separating the chamber into a first portion and a second portion, wherein the ex vivo placenta is housed at least partially in the first portion; and (2) at least one first inlet disposed in the second portion for receiving at least one first tube, the at least one first tube being configured to couple at least one first pump (e.g., at least one syringe) to a fetal compartment of the ex vivo placenta when present in the chamber.

In some embodiments, the system further comprises at least one second inlet disposed in the first portion for receiving at least one second tube, the at least one second tube being configured to couple at least one second pump (e.g., a peristaltic pump) to a maternal compartment of the ex vivo placenta when present in the chamber. In some embodiments, the system further comprises the at least one first tube and the at least one first pump. In some embodiments, the system further comprises the at least one second tube and the at least one second pump. The at least one first pump may be configured to pump a first solution to the fetal compartment of the ex vivo placenta through the at least one first tube. The at least one second pump may be configured to pump a second solution to the maternal compartment of the ex vivo placenta through the at least one second tube. In some embodiments, the system further comprises at least one third tube coupled to an injector (e.g., a contrast power injector) at a first end and to the at least one first tube and/or the at least one second tube at at least one second end.

In some embodiments, the system further comprises the ex vivo placenta. In some embodiments, the system further comprises the MRI device.

In some embodiments, the system further comprises at least one radio frequency (RF) coil arranged proximate to the chamber (e.g., disposed in the second portion) and configured to detect MR signals generated, at least in part, by the ex vivo placenta when present in the chamber during imaging performed by the MRI device. In some embodiments, the chamber further comprises a second partition configured to separate a third portion from the second portions, wherein the third portion comprises the at least one first inlet; and the first portion comprises at least one second inlet for receiving at least one second tube, the at least one second tube being configured to couple at least one second pump to a maternal compartment of the ex vivo placenta when present in the chamber.

According to some aspects of the technology described herein, there is provided a method for perfusing an ex vivo placenta to be imaged using an MRI device, the ex vivo placenta being disposed in a chamber, the method comprising: (1) pumping, using at least one first pump, a solution through at least one first tube to a fetal compartment of the ex vivo placenta; and (2) imaging, using the MRI device, the ex vivo placenta as the solution is pumped through the at least one first tube. In some embodiments, the method further comprises (3) pumping, using at least one second pump, a second solution through at least one second tube to a maternal compartment of the ex vivo placenta.

In some embodiments, the pumping may provide a continuous flow rate. In some embodiments, the pumping may provide a flow rate selected from a range of flow rates between and including a continuous flow rate to a pulsatile flow rate. In some embodiments, pumping using the at least one first pump comprises alternating between a pump off state and a pump on state of the at least one first pump. The alternating may be performed for at least five minutes. The at least one first pump may be alternated between the pump off state and the pump on state at least once per minute. In some embodiments, the alternating is performed continuously for a duration comprising at least a first period of time before the imaging and a second period of time while the imaging is performed.

In some embodiments, the pumping, using the at least one first pump is performed at a first rate and the pumping, using the at least one second pump, is performed at a second rate different than the first rate. In some embodiments, the method further comprises introducing a chemical (e.g., a contrast agent, oxygen, glucose or a therapeutic agent) into the at least one first tube and/or the at least one second tube. For example, an oxygenator may be provided for oxygenating the first and/or second solutions.

In some embodiments, the method further comprises modulating a flow rate of the solution delivered through the at least one first tube to the fetal compartment of the ex vivo placenta by controlling one or more aspects of the pumping by the at least one first pump. For instance, the at least one first pump may be controlled to deliver a pulsatile flow of the solution through the at least one first tube to the fetal compartment of the ex vivo placenta.

According to some aspects of the technology described herein, there is provided a MRI compatible perfusion apparatus, comprising: (1) a chamber configured to house an ex vivo placenta, the chamber comprising (a) at least one first inlet arranged to receive at least one first tube configured to couple to a fetal compartment of the ex vivo placenta when present in the chamber; and (b) at least one second inlet configured to receive at least one second tube configured to couple to a maternal compartment of the ex vivo placenta when present in the chamber; and (2) at least one radio frequency (RF) coil arranged proximate to the chamber and configured to transmit RF signals and/or detect MR signals generated, at least in part, by the ex vivo placenta when present in the chamber during MR imaging.

In some embodiments, the at least one RF coil is coupled to the chamber below a first partition separating a first portion of the chamber from a second portion of the chamber, wherein the ex vivo placenta, when present in the chamber, is disposed at least partially in the first portion. In some embodiments, the chamber further comprises a second partition separating the second portion from a third portion, the first portion comprises the at least one second inlet, and the third portion comprises the at least one first inlet

In some embodiments, the MRI compatible perfusion apparatus further comprises at least one first pump coupled to the at least one first tube; at least one second pump coupled to the at least one second tube; and the at least one first and second tubes. The MRI compatible perfusion apparatus may further comprise a first solution coupled to the at least one first pump and a second solution coupled to the at least one second pump, wherein the second solution is different than the first solution.

According to some aspects of the technology described herein, there is provided a method for generating at least one MR image of an ex vivo placenta disposed in a chamber, the method comprising: (1) perfusing the ex vivo placenta while the chamber is located proximate to at least one RF coil of a MRI device; (2) transmitting, using the at least one RF coil, at least one RF signal; (3) detecting, using the at least one RF coil, at least one MR signal generated, at least in part, by the ex vivo placenta present in the chamber in response to stimulation of the ex vivo placenta by the at least one RF signal; and (4) generating an MR image based on the at least one MR signal detected by the at least one RF coil. In some embodiments, the at least one RF coil is coupled to the chamber such that the at least one RF coil is disposed at least partially below the ex vivo placenta when the ex vivo placenta is present in the chamber during imaging.

In some embodiments, the perfusing comprises: (1) delivering, via at least one first tube, a first solution to a fetal compartment of the ex vivo placenta; and (2) delivering, via at least one second tube, a second solution to a maternal compartment of the ex vivo placenta. Delivering the first solution to the fetal compartment may be performed at a first rate, and delivering the second solution to the maternal compartment may be performed at a second rate different than the first rate.

The aspects and embodiments described above, as well as additional aspects and embodiments, are described further below. These aspects and/or embodiments may be used individually, all together, or in any combination, as the technology is not limited in this respect.

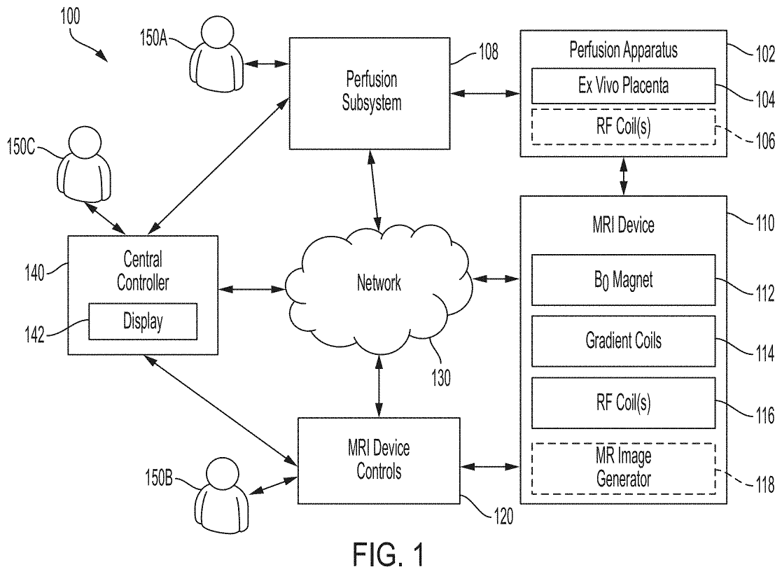

FIG. 1 is a schematic diagram of an example system 100 for imaging an ex vivo placenta using a magnetic resonance imaging device, in accordance with some embodiments described herein. In the illustrative example of FIG. 1, system 100 includes an MRI device 110, MRI device controls 120, a perfusion apparatus 102, a perfusion subsystem 109, and a central controller 140. It should be appreciated that system 100 is illustrative and that a system for imaging an ex vivo placenta may have one or more other components of any suitable type in addition to or instead of the components illustrated in FIG. 1.

As illustrated in FIG. 1, in some embodiments, one or more of the MRI device 110, the perfusion subsystem 108, MRI device controls 120, and/or central controller 140 may be communicatively connected by a network 130. The network 130 may be or include one or more local- and/or wide-area, wired and/or wireless networks, including a local-area or wide-area enterprise network and/or the Internet. Accordingly, the network 130 may be, for example, a hard-wired network (e.g., a local area network within a facility), a wireless network (e.g., connected over Wi-Fi and/or cellular networks), a cloud-based computing network, or any combination thereof. For example, in some embodiments, the MRI device 110 and MRI device controls 120 can be located within a same facility and connected directly to each other or connected to each other via the network 130, while the central controller 140 may be located in a remote facility and connected to the MRI device 110 and/or the MRI device controls 120 through the network 130.

In some embodiments, the MRI system 110 may be configured to perform MR imaging of an ex vivo placenta 104. For example, the MRI system 110 may include a B₀ magnet 112, gradient coils 114, and radio frequency (RF) transmit and receive coils 116 configured to act in concert to perform said MR imaging.

In some embodiments, B₀ magnet 112 may be configured to generate the main static magnetic field, B₀, during MR imaging. The B₀ magnet 112 may be any suitable type of magnet that can generate a static magnetic field for MR imaging. For example, the B₀ magnet 112 may include a superconducting magnet, an electromagnet, and/or a permanent magnet. In some embodiments, the B₀ magnet 112 may be configured to generate a static magnetic field having a particular field strength. For example, the B₀ magnet 112 may be a magnet that can generate a static magnetic field having a field strength of 1.5T, less than 1.5T, or, in some embodiments, a field strength greater than or equal to 1.5T and less than or equal to 3.0T.

In some embodiments, gradient coils 114 may be arranged to provide one or more gradient magnetic fields. For example, gradient coils 114 may be arranged to provide gradient magnetic fields along three substantially orthogonal directions (e.g., x, y, and z). The gradient magnetic fields may be configured to, for example, provide spatial encoding of MR signals during MR imaging. Gradient coils 114 may comprise any suitable electromagnetic coils.

In some embodiments, RF transmit and receive coils 116 may be configured to generate RF pulses to induce an oscillating magnetic field, B1, and/or to receive MR signals from nuclear spins of the imaged subject (e.g., of the fetus 102) during MR imaging. The RF transmit coils may be configured to generate any suitable types of RF pulses useful for performing fetal cardiac MR imaging. RF transmit and receive coils 116 may comprise any suitable RF coils, including volume coils and/or surface coils.

In some embodiments, the MRI system 110 may optionally include MR image generator 118. MR image generator 118 may be configured to generate MR images based on MR data acquired by the MRI system 110 during MR imaging of the fetus 102. For example, in some embodiments, MR image generator 118 may be configured to perform MR image reconstruction to generate MR images in the image domain based on MR data in the spatial frequency domain (e.g., MR data comprising data describing k-space).

As illustrated in FIG. 1, system 100 includes MRI device controls 120 communicatively coupled to the MRI device 110. MRI device controls 120 may be any suitable electronic device configured to send instructions and/or information to MRI device 110, to receive information from MRI device 110, and/or to process obtained MR data. In some embodiments, MRI device controls 120 may be a fixed electronic device such as a desktop computer, a rack-mounted computer, or any other suitable fixed electronic device. A user 150B may interact with the MRI device controls 120 to control aspects of operation of the MRI device 110.

The perfusion apparatus 102 may be configured to receive the ex vivo placenta 104 (e.g., in a chamber, as described herein). For example, the perfusion apparatus 102 may be MRI compatible such that the ex vivo placenta may be imaged by the MRI device 110 when contained by the perfusion apparatus 102. In some embodiments, the perfusion apparatus 102 comprises a chamber for housing the ex vivo placenta, 104, as described herein. In some embodiments, the perfusion apparatus further comprises one or more RF coils 106, for facilitating imaging with the MRI device 110. The RF coil(s) 106 may comprise one or more transmit coils for transmitting at least one RF signal and/or one or more receive coils for detecting at least one MR signal generated, at least in part, by the ex vivo placenta 104 in response to stimulation of the ex vivo placenta 104 by at least one RF signal. In some embodiments, the RF coil(s) 106 may be configured to perform both transmitting and receiving.

The perfusion apparatus 102 may further comprise components configured to facilitate perfusion of the ex vivo placenta 104. For example, as described further herein, the perfusion apparatus 102 may comprise one or more inlets for receiving tubing of a perfusion subsystem 108. The perfusion subsystem 108 may include components for facilitating and controlling perfusion of the ex vivo placenta 104 (e.g., one or more pumps, tubing, and/or perfusate). A user 150A may interact with the perfusion subsystem 108 to control aspects of perfusion of the ex vivo placenta 104 when present in the perfusion apparatus 102.

The system 100 may include a central controller 140 configured to control operation of the system 100. For example, the central controller 140 may be in communication with components of the system 100, such as the perfusion subsystem 108, MRI device 110, and/or MRI device controls 120, directly and/or via network 130. In the illustrated embodiment, the central controller 140 includes a display 142, for example, to display MR data acquired by the MRI device 110. A user 150C may interact with the central controller 140 to control aspects of operation of the system 100.

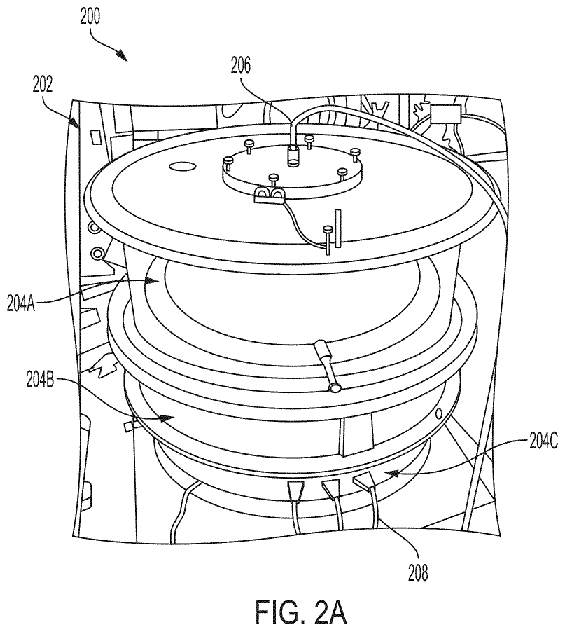

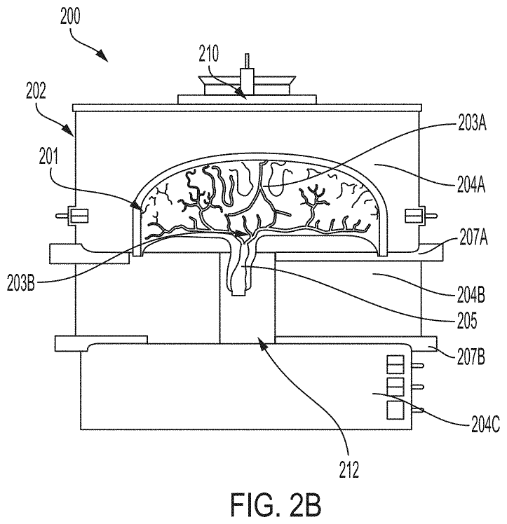

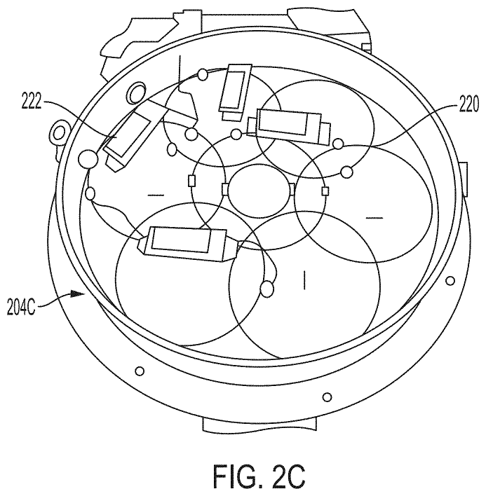

FIG. 2A illustrates an example apparatus 200 for perfusing an ex vivo placenta, in accordance with some embodiments of the technology described herein. The perfusion apparatus 200 may be configured to facilitate perfusion of an ex vivo placenta contained within the apparatus 200. In some embodiments, the perfusion apparatus 200 may comprise features that facilitate imaging the ex vivo placenta with an MRI device, as is further described herein.

As shown in FIG. 2A, the perfusion apparatus 200 comprises a chamber 202 for housing the ex vivo placenta. In some embodiments, the chamber 202 provides a temperature-controlled environment for the ex vivo placenta. The chamber 202 may isolate the ex vivo placenta from the MRI device, such that inserting and removing the ex vivo placenta from an imaging region of the MRI device is made easier, without needing to decontaminate the MRI device after imaging is performed.

The chamber 202 may comprise multiple portions 204A-C. As further described herein, a first portion 204A may be separated from a second portion 204B by a first partition 207A, as is further shown in FIG. 2B. The second portion 204B may be separated from a third portion 204B by a second partition 207B, as is further shown in FIG. 2B.

FIG. 2A further illustrates tubing 206, 208 which may be inserted into the chamber 202. For example, tubing 206, 208 may deliver perfusate to maternal and fetal compartments of the ex vivo placenta, respectively. The chamber 202 may comprise inlets for receiving the tubing 206, 208.

FIG. 2B illustrates a cross-sectional view of the example apparatus of FIG. 2A, in accordance with some embodiments of the technology described herein. As shown in FIG. 2B, an ex vivo placenta 201 is contained in a first portion 204A of the chamber 202. The first portion 204A further comprises an inlet 210 for receiving tubing 206. The tubing 206 may be coupled to a maternal compartment 203A of the ex vivo placenta 201 by one or more catheters for delivering perfusate to the maternal compartment 203A of the ex vivo placenta. A third portion 204C of the chamber 202 may comprise an inlet 212 for receiving tubing 208. The tubing 208 may be coupled to a fetal compartment 203B of the ex vivo placenta 201, for example, through an umbilical cord 205 of the ex vivo placenta 201 by one or more catheters for delivering perfusate to the fetal compartment 203B of the ex vivo placenta 201.

As is further shown in FIG. 2C, a second portion 204B of the chamber 202 may comprise one or more RF coils 220 configured to facilitate imaging of the ex vivo placenta 201 with an MRI device. The one or more RF coils 220 may be disposed at least partially below the ex vivo placenta 201, which may increase a signal-to-noise ratio of MR signals sensed by the MRI device. As described herein, the RF coil(s) 220 may comprise one or more transmit coils for transmitting at least one RF signal and/or one or more receive coils for detecting at least one MR signal generated, at least in part, by the ex vivo placenta 201 in response to stimulation of the ex vivo placenta 201 by at least one RF signal. In some embodiments, the RF coil(s) 220 may be configured to perform both transmitting and receiving. The chamber 202 may further comprise circuitry 222 (e.g., one or more circuit boards) configured to control the RF coil(s) 220.

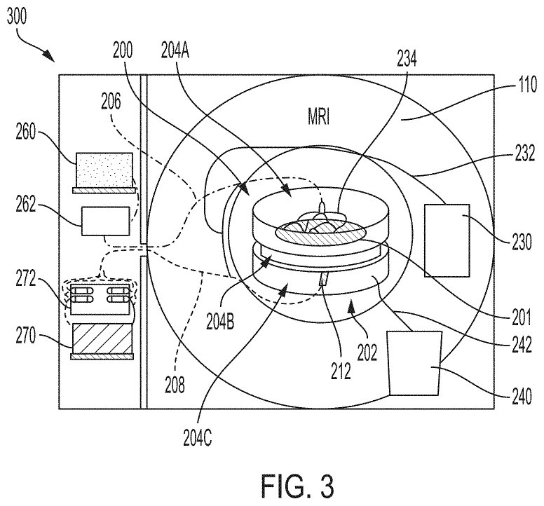

FIG. 3 illustrates an example system 300 for perfusing an ex vivo placenta, in accordance with some embodiments of the technology described herein. In the illustrated embodiments, the system 300 comprises the perfusion apparatus 200 and one or more additional components for facilitating perfusion of the ex vivo placenta 201. In some embodiments, the system 300 further comprises the MRI device 110.

For example, system 300 may comprise one or more pumps for delivering perfusate to maternal and fetal compartments of the ex vivo placenta 201. As described herein, the system 300 may be configured to perfuse the respective compartments of the ex vivo placenta independently. As such, perfusate from a first reservoir 260 may be delivered via first tubing 206 to the maternal compartment of the ex vivo placenta 201 using a first pump 262. Likewise, perfusate from a second reservoir 270 may be delivered via second tubing 208 to the fetal compartment of the ex vivo placenta 201 using a second pump 272.

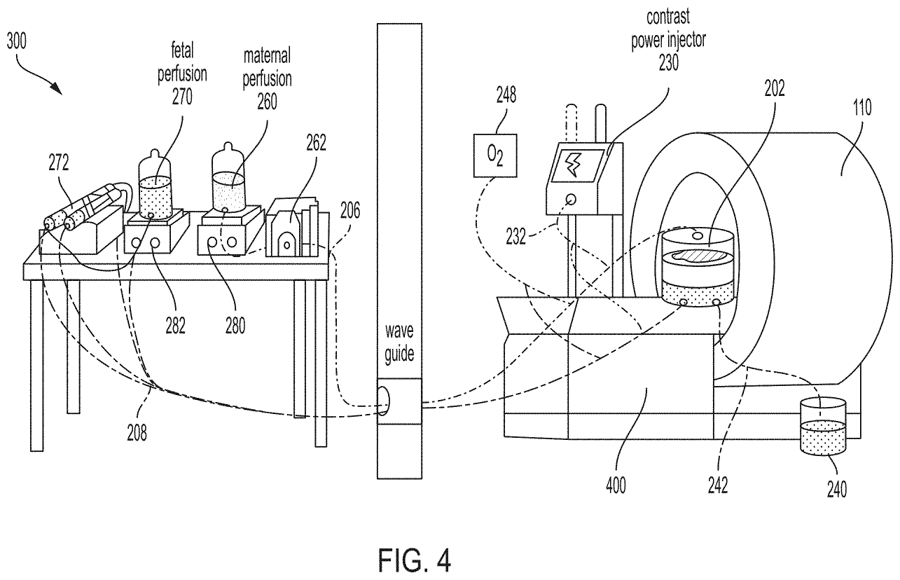

The perfusate delivered to the ex vivo placenta 201 may be any suitable composition. For example, in some embodiments, the perfusate comprises a mixture of glucose, buffered saline, and nitroglycerine. In some embodiments, the first and second reservoirs 260, 270 of perfusate comprise different compositions. The perfusate may be selected to inhibit degradation of the ex vivo placenta. As shown in FIG. 4, the first and second reservoirs 260, 270 of perfusate may be disposed on respective warming plates 280, 282, for warming the reservoirs 260, 270.

The first and second pumps 262, 272 may be configured to deliver the perfusate at different flow rates. In some embodiments, the first and/or second pump 262, 272 may be configured to provide a constant flow of perfusate to the ex vivo placenta 201. In some embodiments, a flow rate of perfusate delivered to the maternal compartment of the ex vivo placenta and/or to the fetal compartment of the ex vivo placenta may be modulated. In some embodiments, the first pump 262 may comprise a peristaltic pump. In some embodiments, the second pump 272 may comprise one or more syringes. In some embodiments, the first and/or second pump may be configured to introduce pulsatility into perfusate flow to the ex vivo placenta 201. For example, the second pump 272 may be configured to introduce pulsatility into the perfusate flowing to the fetal compartment of the ex vivo placenta 201 from the second reservoir 270 (e.g., by controlling a flow rate and/or pump on/off state).

The first and second tubing 206, 208 may be coupled to one or more catheters for delivering the perfusate to respective compartments of the ex vivo placenta 201. As shown in FIG. 3, one or more catheters 234 are coupled to the first tubing 206 for delivering perfusate to the maternal compartment of the ex vivo placenta 201. Although not shown in FIG. 3, one or more catheters may likewise be coupled to the second tubing 208 via the inlet 212 for delivering perfusate to the fetal compartment of the ex vivo placenta. Placement of the catheters into the ex vivo placenta 201 may be designed to mimic physiological spiral arteries and allow perfusate to run through veins of the ex vivo placenta 201.

In some embodiments, the system 300 further comprises a third tubing 232 for delivering a chemical to the first and/or second tubing 206, 208. In some embodiments, the chemical may comprise a contrast enhancer, such as a gadolinium, for enhancing the contrast of acquired MR images or other chemicals such as oxygen, glucose or a therapeutic agent. The third tubing 232 may be coupled to a power injector 230 for delivering the contrast enhancer to the first and/or second tubing 206, 208.

In some embodiments, the system 300 further comprises a fourth tubing 242 for removing waste from the chamber. As shown in FIG. 3, a fourth tubing 242 is coupled to the third compartment 204C of the chamber 202 for extracting waste from the chamber 202 and into an overflow compartment 240.

FIG. 4 illustrates additional aspects of the example system of FIG. 3, in accordance with some embodiments of the technology described herein. For example, FIG. 4 illustrates placement of the chamber 202 relative to an MRI device 110. As shown in FIG. 3, the chamber 202 may be placed on a base 400 of an MRI device 110, within an imaging region of the MRI device 110.

The inventors have recognized that there are challenges in preserving a biological perfusion phantom developed from an ex vivo organ. Aspects of the system 300 may be designed to inhibit degradation of the ex vivo placenta 201 and increase the length of time it is usable as a perfusion phantom. For example, as described herein, high quality perfusate, which may be temperature controlled, may be delivered to the ex vivo placenta 201. The chamber 202 may be temperature controlled and provide oxygen, glucose and/or a therapeutic agent for the ex vivo placenta 201. Leakage from the ex vivo placenta may be controlled, as described herein, with additional tubing coupled to the chamber 202. Such features may enable use of the ex vivo placenta for at least 4-6 hours, and even up to 24 hours, in some embodiments.

As described herein, in some embodiments, the system may further comprise at least one oxygenator 248 for modulating the oxygenation of the solutions delivered to the ex vivo placenta 201. For example, the oxygenator 248 may deliver oxygen to the solution(s) delivered to the ex vivo placenta and may remove carbon dioxide from the solution(s). In some embodiments, the oxygenator may deliver oxygen to the solution delivered to the maternal compartment of the ex vivo placenta 201 via first tubing 206, to the solution delivered to the fetal compartment of the ex vivo placenta via second tubing 208, or both. In some embodiments, the rate of oxygenation of the respective solutions delivered to the maternal and fetal compartments of the ex vivo placenta 201 may be the same and, in other embodiments, the rate of oxygenation of the respective solutions may be different.

The inventors have developed techniques for perfusing and imaging the ex vivo placenta using an MRI device. In some embodiments, the ex vivo placenta is prepared for imaging by washing the placenta and refrigerating the placenta until imaging is performed. When it is desired to perform imaging, the placenta may be placed in the controlled environment of the chamber, with the umbilical cord extending down towards a bottom of the chamber. Catheters may be coupled to the maternal and/or fetal compartments of the placenta mimicking arterial layout. Once the placenta is placed in the chamber and the catheters have been coupled to the placenta, perfusion and imaging of the ex vivo placenta may be performed.

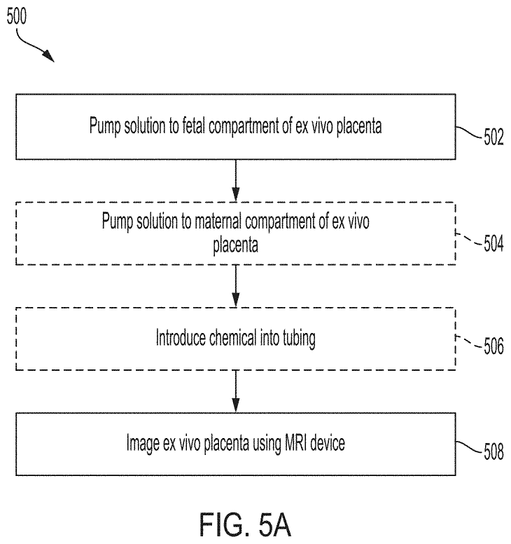

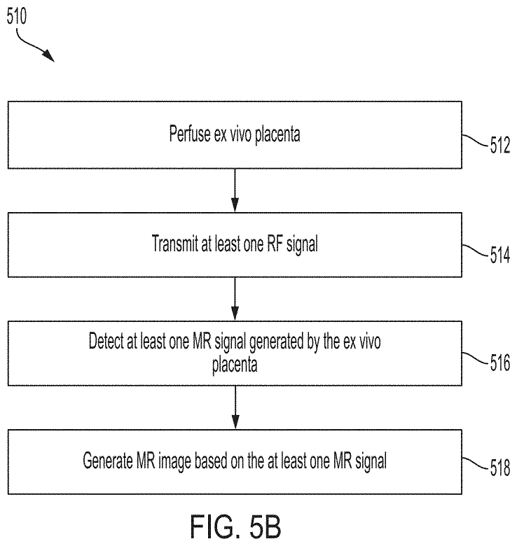

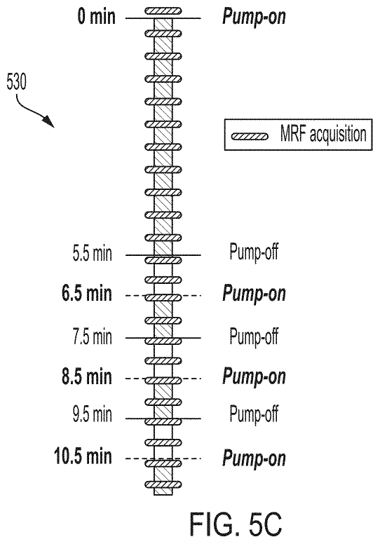

FIG. 5A illustrates an example process 500 for perfusing an ex vivo placenta, in accordance with some embodiments of the technology described herein. Process 500 begins at act 502 where a solution (e.g., perfusate) is pumped to a fetal compartment of the ex vivo placenta. For example, at least one pump (e.g., one or more syringes) may be used to pump perfusate from a reservoir through tubing and/or one or more catheters, and to the fetal compartment of the ex vivo placenta. The pumping may be controlled to achieve a desired flow rate and/or pulsatility of flow.

In some embodiments, the process 500 may include act 504. At act 504, a solution (e.g., perfusate) is pumped to a maternal compartment of the ex vivo placenta. For example, at least one pump (e.g., a peristaltic pump) may be used to pump perfusate from a reservoir through tubing and/or one or more catheters, and to the maternal compartment of the ex vivo placenta. The pumping may be controlled to achieve a desired flow rate. In some embodiments, an orientation of the catheter(s) coupling the tubing to the maternal compartment of the placenta may be arranged to achieve a particular direction of flow (e.g., anterior to posterior, posterior to anterior, etc.) by controlling the inflow direction of the perfusate. In some embodiments, one or more catheters may be arranged such that a direction of the flow may be changed without needing to move the placenta. In some embodiments, the perfusion apparatus may be configured to control a simulated heart rate in the maternal compartment of the ex vivo placenta.

The process 500 may optionally proceed to act 506, where a chemical is introduced into a tubing. In some embodiments, the chemical may be a contrast enhancer, such as gadolinium, oxygen, glucose or a therapeutic agent introduced into the perfusate flow via tubing coupled to the fetal and/or maternal compartment.

At act 508, the ex vivo placenta may be imaged using the MRI device. In some embodiments, the imaging may be performed while the ex vivo placenta is being perfused. In some embodiments, the imaging may be performed, at least in part, with an RF coil coupled to the chamber containing the ex vivo placenta.

FIG. 5B illustrates an example process 510 for generating at least one magnetic resonance image of an ex vivo placenta, in accordance with some embodiments of the technology described herein. Process 510 begins at act 512, where the ex vivo placenta is perfused. For example, a fetal and/or maternal compartment of the ex vivo placenta may be perfused with perfusate delivered via one or more catheters and tubing from a perfusate reservoir using at least one pump. Perfusing the fetal compartment, maternal compartment and/or the intervillous space may comprise turning at least one pump on. The at least one pump may be turned on according to a step function, a ramp function, a non-linear function, and/or in any other suitable manner.

At act 514, at least one RF signal may be transmitted to the ex vivo placenta. In particular, the at least one RF signal may be transmitted by at least one RF coil. In some embodiments, the at least one RF coil is part of an MRI device. In some embodiments, the at least one RF coil is disposed within a chamber containing the ex vivo placenta, proximate to the ex vivo placenta (e.g., disposed at least partially below the ex vivo placenta).

At act 516, at least one MR signal generated by the ex vivo placenta in response to stimulation by the at least one RF signal may be detected. For example, the at least one MR signal may be detected by at least one RF coil. The at least one RF coil may be the same coil(s) or a different coil than the at least one RF coil which transmitted the at least one RF signal. In some embodiments, the at least one RF coil which detects the at least one MR signal is part of the MRI device. In some embodiments, the at least one RF coil which detects the at least one MR signal is disposed within a chamber containing the ex vivo placenta, proximate to the ex vivo placenta (e.g., disposed at least partially below the ex vivo placenta).

At act 518, at least one MR image may be generated based on the at least one MR signal. The at least one MR image may be used, for example, to study the effects of flow on MR parameters.

FIG. 5C illustrates an example timing diagram for alternating between pump on and pump off states, in accordance with some embodiments of the technology described herein. According to some aspects of the technology provided herein, MR data may be collected while the ex vivo placenta is perfused, in order to study the effects of flow on MR parameters. In some embodiments, perfusing the fetal and/or maternal compartment of the ex vivo placenta may comprise alternating one or more pumps between a pump off state and a pump on state.

As shown in FIG. 5C, the one or more pumps may be turned to a pump on state for an initial period of time. In particular, the one or more pumps are turned on for an amount of time (e.g., at least five minutes) to allow flow of perfusate through the placenta to reach a steady state. MR data (e.g., MRF date) may be acquired at intervals during the pumping. After reaching the steady state of flow, the one or more pumps may alternate between the pump on state and the pump off state in intervals (e.g., at least once per minute). MR data may be acquired throughout this period of alternating between pump on/off states. For example, MR data may be acquired shortly after a change in pump state (e.g., 3 seconds after), and again at a later time after the change in pump state (e.g., 30 seconds after).

After sufficient MR data is acquired, the one or more pumps may be turned to a pump off state to stop flow of perfusate to the ex vivo placenta. In some embodiments, one or more of the methods described herein may be repeated as desired. In some embodiments, a vascular cast of the ex vivo placenta may be obtained for further information on the structure of the placenta.

As described herein, the inventors have recognized that the placental perfusion phantom described herein more precisely mimics biological perfusion than any previous known phantom and as such may be used to develop and validate MRI perfusion quantification and to examine the confounding effects of perfusion on quantitative MRI techniques. In some embodiments, the biological perfusion phantom may be used to evaluate new MRI approaches, such as developing new pulse sequences, and/or calibrating via MRF (e.g., by analyzing T1 and T2 mappings to evaluate O₂ exchange). In some embodiments, MR data acquired from scanning the biological perfusion phantom may be compared to a library containing different tissue parameter sets. In some embodiments, the biological perfusion phantom may be used for performing arterial spin labeling (ASL). In some embodiments, the MR data acquired using the biological perfusion phantom may be used to inform the study of other anatomy, such as the brain, for example, for which ex vivo imaging is not possible.

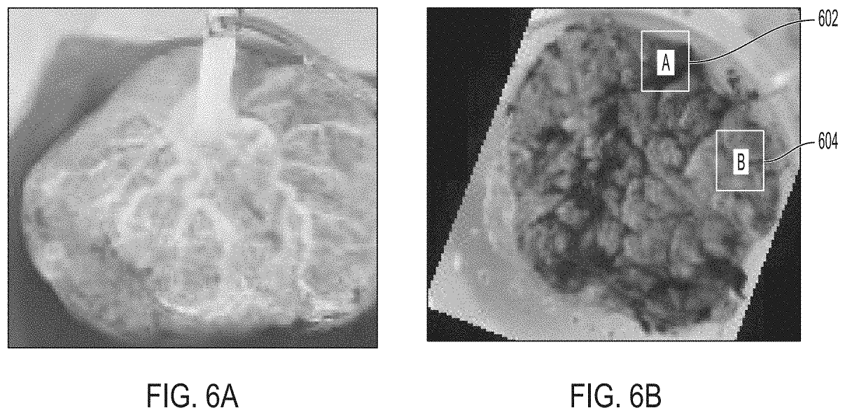

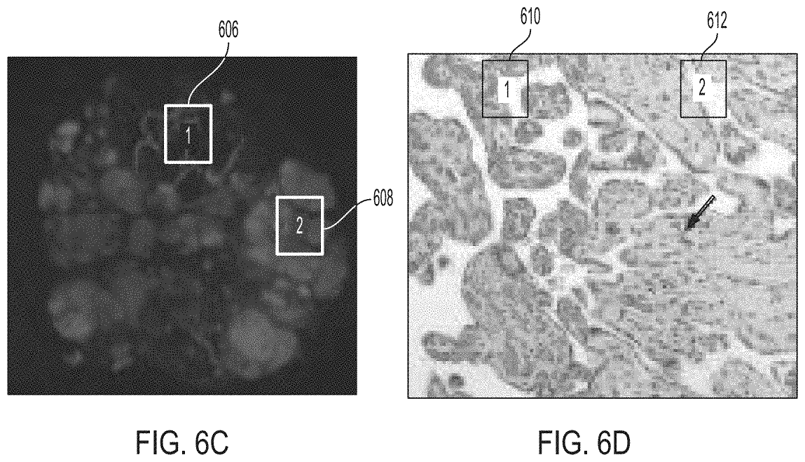

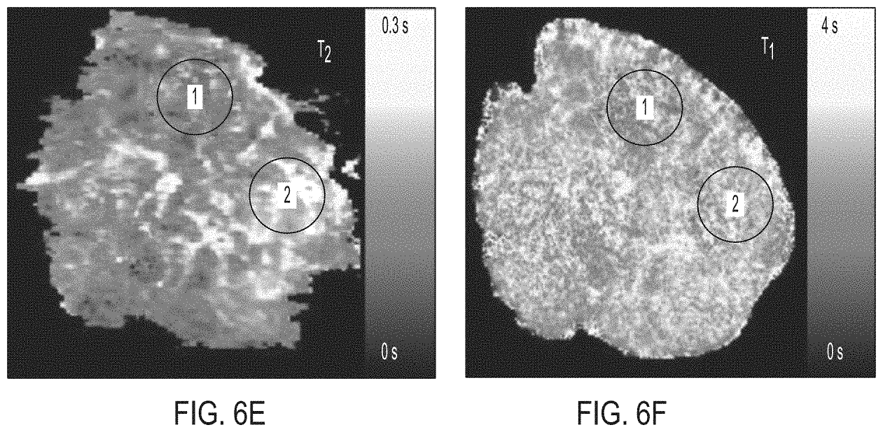

FIGS. 6A-12B illustrate example data acquired according to the techniques described herein. FIG. 6A illustrates an example of an ex vivo placenta, in accordance with some embodiments of the technology described herein. FIG. 6B illustrates an example of a perfused ex vivo placenta, in accordance with some embodiments of the technology described herein. Region A 602 illustrates an area which remains red after perfusion and region B 604 illustrates an area appearing yellow/white.

FIG. 6C illustrates an example magnetic resonance angiography maximum intensity projection of the ex vivo placenta of FIG. 6A during the parenchymal phase of the contrast passage, captured using imaging of a biological placental perfusion device configured in accordance with some embodiments of the technology described herein. Regions 1 and 2 606, 608 illustrate areas of lesser and greater, respectively, enhancement. FIG. 6D illustrates an example histological examination of portions of the ex vivo placenta of FIG. 6A. Region 1 610 illustrates the less enhanced area shown in FIG. 6C, while region 2 612 illustrates the more enhanced area of FIG. 6C. Region 1 610 illustrates no yellow dye and red blood cell congestion throughout the villous tree. Region 2 612 illustrates dye in most of the terminal villi and more open vasculature. FIG. 6E illustrates an example T2 map of the ex vivo placenta of FIG. 6A. FIG. 6F illustrates an example T1 map of the ex vivo placenta of FIG. 6A.

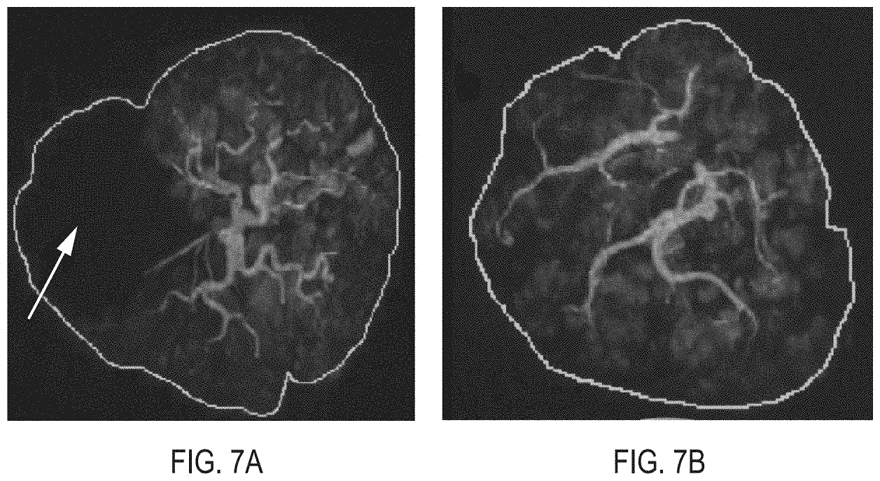

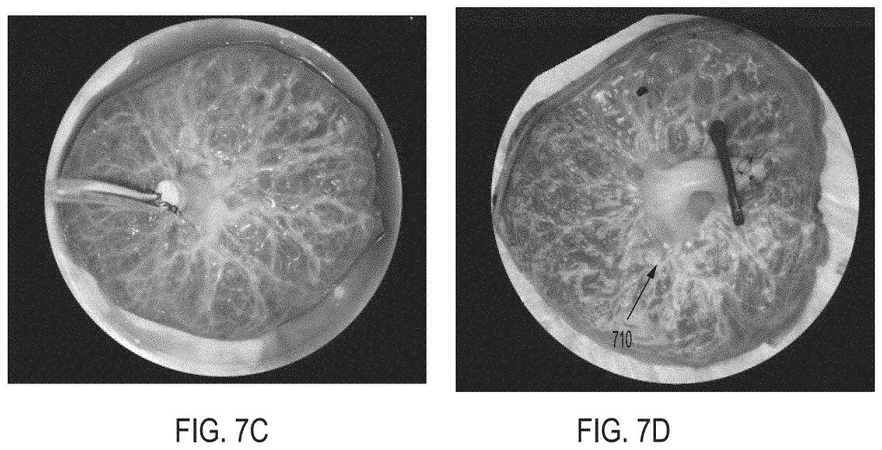

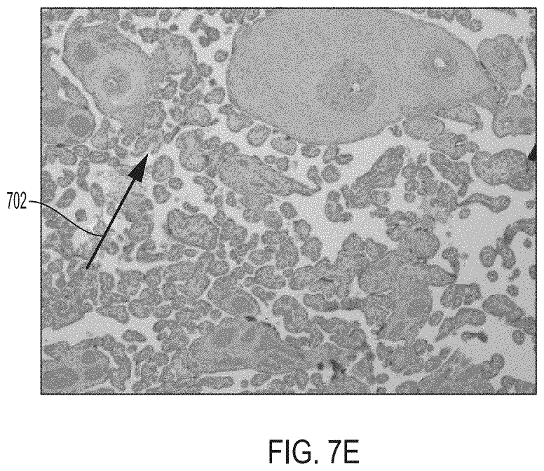

FIGS. 7A-7B illustrate example contrast enhanced magnetic resonance angiography projections of an ex vivo placenta, captured using imaging of a biological placental perfusion device configured in accordance with some embodiments of the technology described herein. FIG. 7A illustrates a representative contrast enhanced magnetic resonance angiography after contrast passage where there is a region of no enhancement in the placenta. Structural imaging may provide a boundary of the placenta shown in FIG. 7A. FIG. 7B illustrates a representative magnetic resonance angiography after contrast passage where the entire placenta shows uniform enhancement in comparison to FIG. 7A. The placenta shown in FIG. 7B was obtained from a pregnant subject with intrauterine growth restriction.

FIG. 7C illustrates an example ex vivo placenta after a washout has been performed, that may be used with a biological placental perfusion device configured in accordance with some embodiments of the technology described herein. As shown in FIG. 7C, after washout of the umbilical artery, the arteries of the placenta appear translucent. FIG. 7D illustrates the example ex vivo placenta of FIG. 7C after magnetic resonance imaging has been performed. As shown in FIG. 7D, after scanning, yellow tissue dye 710 is visible in the arteries contrast enhanced feeding regions, and not in regions of no contrast enhancement. FIG. 7E illustrates an example histological examination of portions of the ex vivo placenta of FIG. 7C. FIG. 7E illustrates a region 702 of villous congestion.

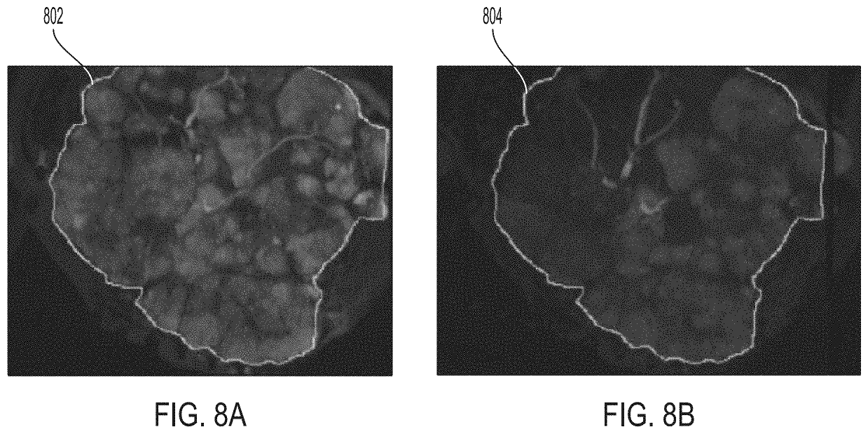

FIGS. 8A-8B illustrate example images of a perfused ex vivo placenta, captured using imaging of a biological placental perfusion device configured in accordance with some embodiments of the technology described herein. FIG. 8A illustrates perfusion of the umbilical artery. FIG. 8B illustrates perfusion of the umbilical vein. FIGS. 8A-8B illustrate outlines, 802, 804 reflecting the extent of umbilical vein perfusion contrast enhancement. It can be seen that a large area of the terminal villi of the placenta is perfused by the umbilical vein and not by the umbilical artery.

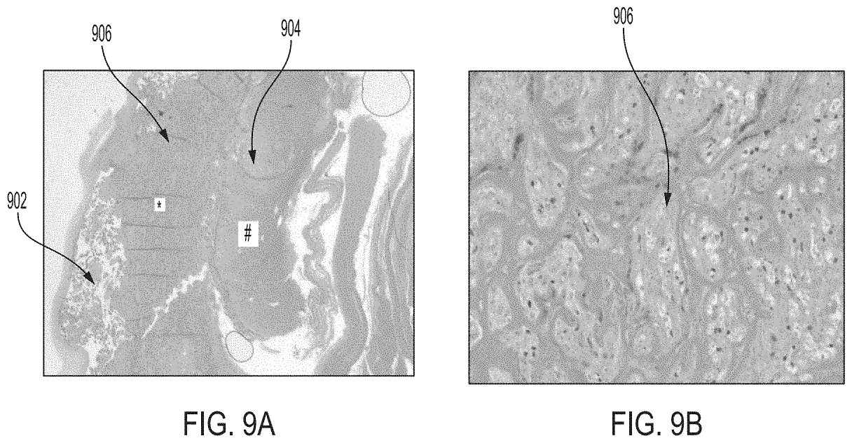

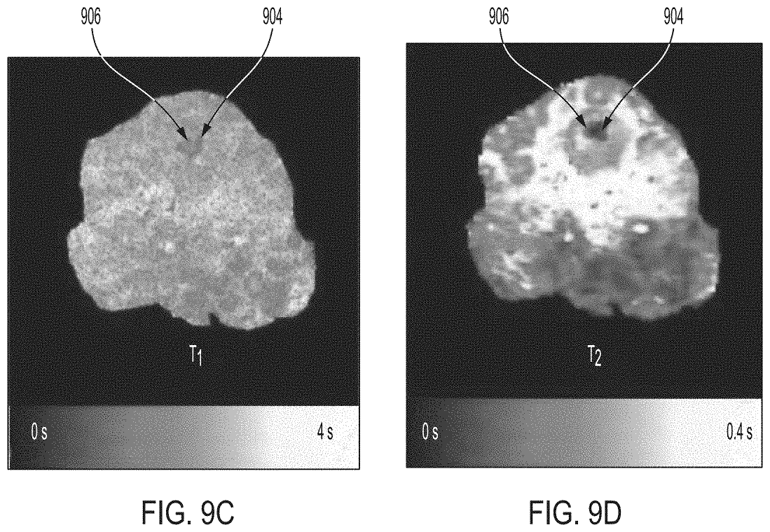

FIGS. 9A-9B illustrate images of an ex vivo placenta having infarcted regions, captured using imaging of a biological placental perfusion device configured in accordance with some embodiments of the technology described herein. As shown in FIGS. 9A-9B, an intervillous thrombus 904 was detected during histopathological examination of the placenta. An associated infarction 906 illustrated as perivillous fibrin around the thrombus is also shown. Region 902 illustrates normal intervillous tissue. FIG. 9C illustrates an example T1 map of the ex vivo placenta of FIGS. 9A-9B, in accordance with some embodiments of the technology described herein. FIG. 9D illustrates an example T2 maps of the ex vivo placenta of FIGS. 9A-9B. The intervillous thrombus and surrounding infarct correspond to an abnormal area shown in the relaxometry maps in FIGS. 9C-9D. The low T1 and T2 values in the lower half of the placenta shown in FIGS. 9C-9D correlate with villi congested by red blood cells which may result in incomplete washout in that area.

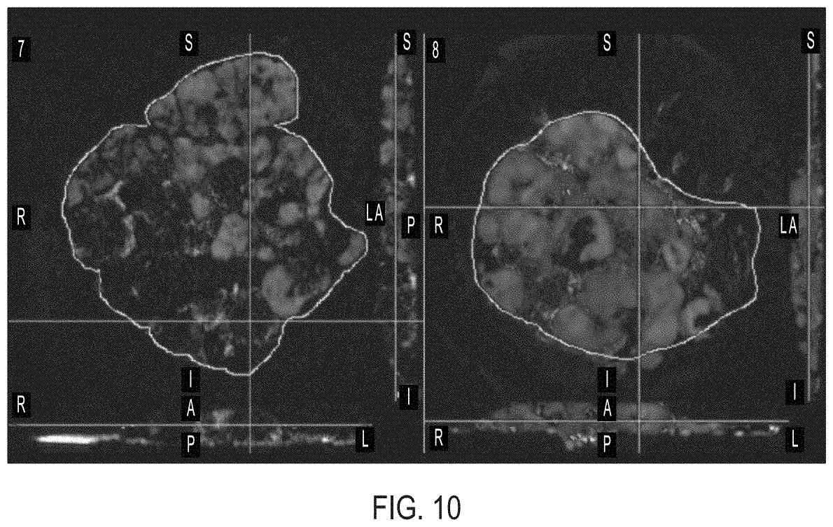

FIG. 10 illustrates example magnetic resonance angiography data during perfusion of an intervillous space and umbilical artery of an ex-vivo placenta, captured using imaging of a biological placental perfusion device configured in accordance with some embodiments of the technology described herein.

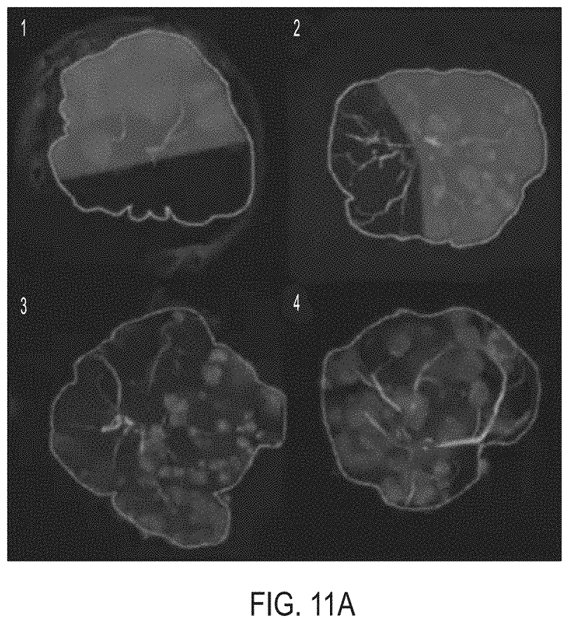

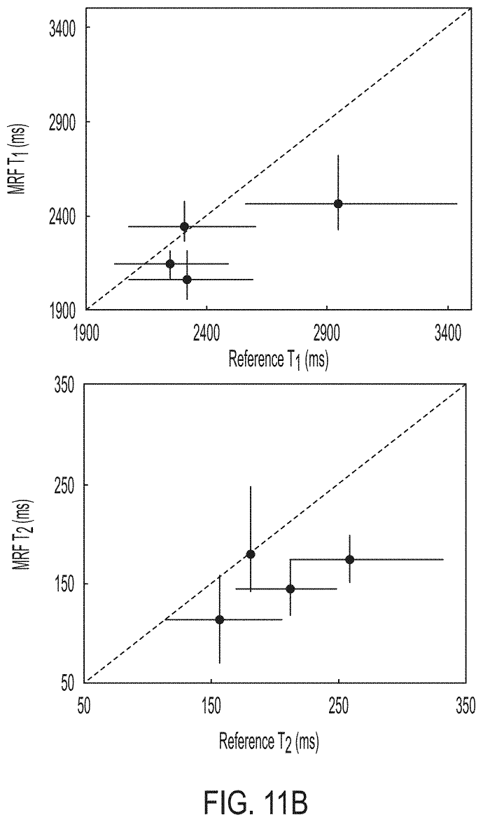

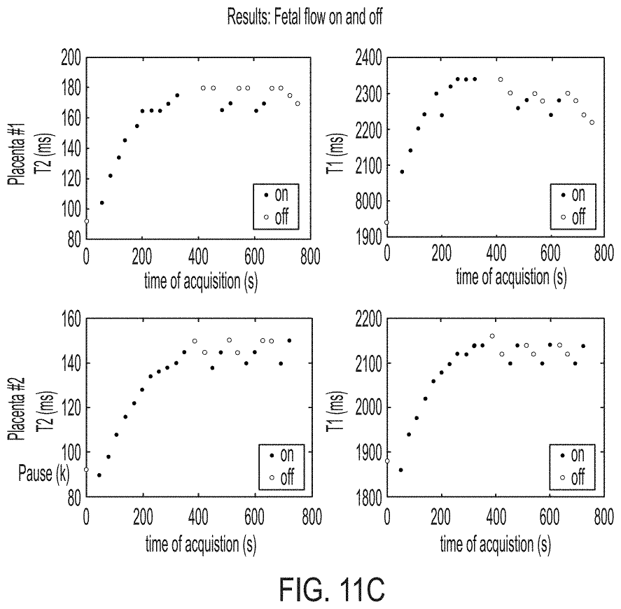

FIG. 11A illustrates sine coronal maximum intensity projections of four perfused ex vivo placentas, determined based on imaging data captured using imaging of a biological placental perfusion device configured in accordance with some embodiments of the technology described herein. Perfused region maps are overlaid onto the projections, which illustrate well perfused regions on right portions of the projections. FIG. 11B illustrates example correlation plots between magnetic resonance fingerprinting for the projections of FIG. 11A and reference techniques for T1 and T2. FIG. 11C illustrate example T1 and T2 graphs of the ex vivo placentas of FIG. 11A.

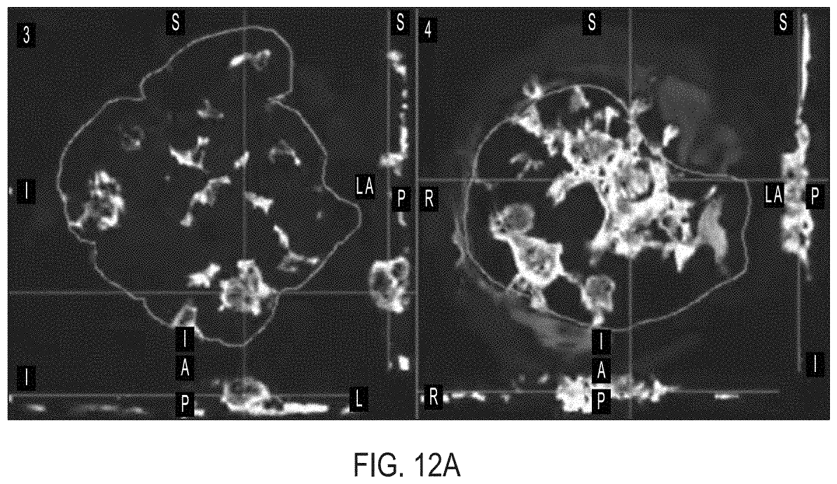

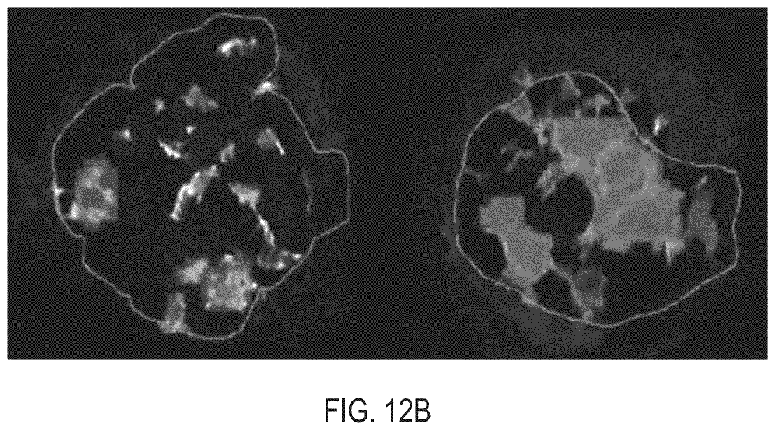

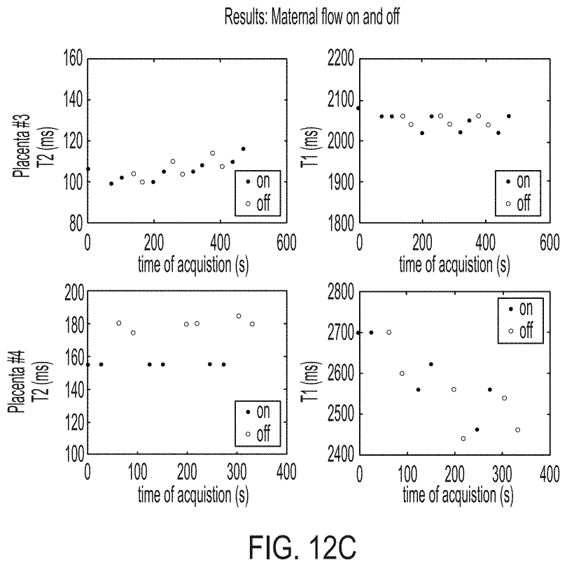

FIG. 12A illustrates example magnetic resonance images obtained during perfusion of maternal compartments of a pair of ex vivo placentas with the biological placental perfusion device configured in accordance with some embodiments of the technology described herein. FIG. 12B illustrates the example magnetic resonance images of FIG. 12A. FIG. 12C illustrates example T1 and T2 graphs of the ex vivo placentas of FIG. 12A.

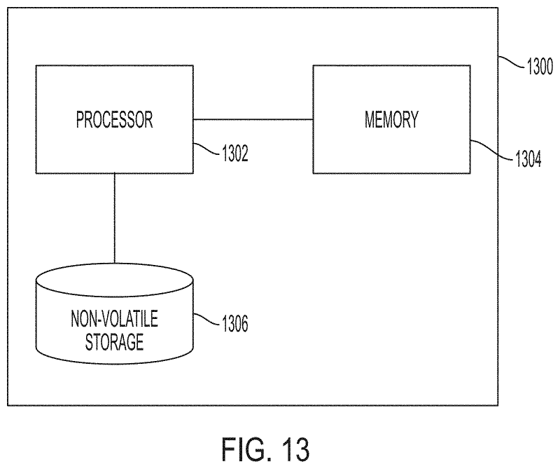

FIG. 13 shows a block diagram of an example computer system 1300 that may be used to implement embodiments of the technology described herein. The computing device 1300 may include one or more computer hardware processors 1302 and non-transitory computer-readable storage media (e.g., memory 1304 and one or more non-volatile storage devices 1306). The processor(s) 1302 may control writing data to and reading data from (1) the memory 1304; and (2) the non-volatile storage device(s) 1306. To perform any of the functionality described herein, the processor(s) 1302 may execute one or more processor-executable instructions stored in one or more non-transitory computer-readable storage media (e.g., the memory 1304), which may serve as non-transitory computer-readable storage media storing processor-executable instructions for execution by the processor(s) 1302.

Having thus described several aspects and embodiments of the technology set forth in the disclosure, it is to be appreciated that various alterations, modifications, and improvements will readily occur to those skilled in the art. For example, although examples are provided herein for use of the biological perfusion phantom with an MRI device, it should be appreciated that the systems and methods described herein may be used in combination with any suitable device, and are not limited to MRI. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated. that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A system for perfusing an ex vivo placenta to be imaged using a magnetic resonance imaging (MRI) device, the system comprising:
a chamber configured to house the ex vivo placenta therein, wherein the chamber includes a first partition separating the chamber into a first portion and a second portion, wherein the ex vivo placenta is housed at least partially in the first portion; and
at least one first inlet disposed in the second portion for receiving at least one first tube, the at least one first tube being configured to couple at least one first pump to a fetal compartment of the ex vivo placenta when present in the chamber.

2. The system of claim 1, further comprising:
at least one second inlet disposed in the first portion for receiving at least one second tube, the at least one second tube being configured to couple at least one second pump to a maternal compartment of the ex vivo placenta when present in the chamber.

3. The system of claim 1, further comprising the at least one first tube and the at least one first pump.

4. The system of claim 2, further comprising the at least one second tube and the at least one second pump.

5. The system of claim 1, further comprising at least one radio frequency (RF) coil arranged proximate to the chamber and configured to detect MR signals generated, at least in part, by the ex vivo placenta when present in the chamber during imaging performed by the MRI device.

6. The system of claim 5, wherein the at least one RF coil is disposed in the second portion.

7. The system of claim 4, wherein:
the at least one first pump is configured to pump a first solution to the fetal compartment of the ex vivo placenta through the at least one first tube; and
the at least one second pump is configured to pump a second solution to the maternal compartment of the ex vivo placenta through the at least one second tube.

8. The system of claim 1, further comprising:
at least one third tube coupled to an injector at a first end and to the at least one first tube and/or the at least one second tube at at least one second end.

9. The system of claim 8, wherein the injector comprises an oxygenator for oxygenating the first and/or second solutions.

10. A method for perfusing an ex vivo placenta to be imaged using a magnetic resonance imaging (MRI) device, the ex vivo placenta being disposed in a chamber, the method comprising:
pumping, using at least one first pump, a solution through at least one first tube to a fetal compartment of the ex vivo placenta; and
imaging, using the MRI device, the ex vivo placenta as the solution is pumped through the at least one first tube.

11. The method of claim 10, further comprising pumping, using at least one second pump, a second solution through at least one second tube to a maternal compartment of the ex vivo placenta.

12. The method of claim 10, wherein pumping, using the at least one first pump, comprises alternating between a pump off state and a pump on state of the at least one first pump.

13. The method of claim 10, wherein the pumping provides a flow rate selected from a range of flow rates between and including a continuous flow rate to a pulsatile flow rate.

14. The method of claim 12, wherein the alternating is performed for at least five minutes.

15. The method of claim 12, wherein the at least one first pump is alternated between the pump off state and the pump on state at least once per minute.

16. The method of claim 12, wherein the alternating is performed continuously for a duration comprising at least a first period of time before performing the imaging and a second period of time while the imaging is performed.

17. The method of claim 11, wherein the pumping, using the at least one first pump, is performed at a first rate and the pumping, using the at least one second pump, is performed at a second rate different than the first rate.

18. The method of claim 11, further comprising introducing a chemical into the at least one first tube and/or the at least one second tube.

19. The method of claim 18, wherein the chemical comprises a contrast agent.

20. The method of claim 10, wherein the pumping comprises:
delivering, via the least one first tube, the solution to a fetal compartment of the ex vivo placenta; and
delivering, via at least one second tube, a second solution to a maternal compartment of the ex vivo placenta.

21. The method of claim 20, wherein:
delivering the solution to the fetal compartment is performed at a first rate; and
delivering the second solution to the maternal compartment is performed at a second rate different than the first rate.

22. A magnetic resonance imaging (MRI) compatible perfusion apparatus comprising:
a chamber configured to house an ex vivo placenta therein, the chamber comprising:
at least one first inlet arranged to receive at least one first tube configured to couple to a fetal compartment of the ex vivo placenta when present in the chamber; and
at least one second inlet configured to receive at least one second tube configured to couple to a maternal compartment of the ex vivo placenta when present in the chamber; and
at least one radio frequency (RF) coil arranged proximate to the chamber and configured transmit RF signals and/or detect MR signals generated, at least in part, by the ex vivo placenta when present in the chamber during MR imaging.

23. The MRI compatible perfusion apparatus of claim 22, wherein the at least one RF coil is coupled to the chamber below a first partition separating a first portion of the chamber from a second portion of the chamber, wherein the ex vivo placenta, when present in the chamber, is disposed at least partially in the first portion.

24. The MRI compatible perfusion apparatus of claim C3, further comprising:
a first solution coupled to at least one first pump, the at least one first pump being coupled to the first tube; and
a second solution coupled to at least one second pump, the at least one second pump being coupled to the at least one second tube, wherein the second solution is different than the first solution.

25. The MRI compatible perfusion apparatus of claim 23, wherein the chamber further comprises a second partition separating the second portion from the third portion, the first portion comprises the at least one second inlet, and the third portion comprises the at least one first inlet.
